(19) United States
(12) Patent Application Publication
(10) Pub. No.: US 2007/0071725 A1
(43) Pub. Date: Mar. 29, 2007

(54) Nitric oxide synthase gene transfer

(76) Inventor: David James Paterson, Oxford (GB)

Correspondence Address: PEARL COHEN ZEDEK, LLP;PEARL COHEN ZEDEK LATZER, LLP, 1500 BROADWAY 12TH FLOOR, NEW YORK, NY, 10036, US

(21) Appl. No.: 11/589,992

(22) Filed: Oct. 31, 2006

Related U.S. Application Data
(63) Continuation-in-part of application No. 10/534,888, filed on Sep. 27, 2005.

(30) Foreign Application Priority Data
Nov. 13, 2002 (GB) GB 0226463.8

(51) Int. Cl. A61K 48/00; C12N 15/867; C12N 15/861; C12N 7/02

(52) U.S. Cl. 424/93.2; 435/456; 977/802; 435/239

(57) ABSTRACT

This invention relates to to gene transfer, specifically, provided herein are methods, vectors and compositions for the transfer of nNOS gene affecting overexpression of nNOS in sympathetic and parasympathetic nervous system and its subsequent use in the treatment of related pathologies.

### CROSS-REFERENCE TO RELATED APPLICATIONS

This application is a Continuation-in-part application of U.S. patent application Ser. No. 10/534,888, filed Sep. 27, 2005, which is a National Phase Application of PCT International Application No. PCT/GB2003/004934, International Filing Date 13 Nov. 2003, claiming priority of Patent Application, GB 0226463.8, filed 13 Nov. 2002, all which are incorporated herein by reference in their entirety,

### FIELD OF INVENTION

The invention relates to gene transfer, specifically, provided herein are methods, vectors and compositions for the transfer of nNOS gene affecting overexpression of nNOS in sympathetic and parasympathetic nervous system and its subsequent use in the treatment of related pathologies.

### BACKGROUND OF THE INVENTION

Selective targeting of cardiac sympathetic neurons is an important step in developing a novel therapeutic anti-adrenergic strategy, since sympathetic hyper-responsiveness is a feature of cardiovascular diseases. Gene transfer techniques with viral vectors expressing nitric oxide synthases (NOS) have been successfully used to demonstrate the potential signaling role of the biological messenger nitric oxide (NO) in regulating endothelial function, ventricular myocyte calcium handling and autonomic neurotransmission. Viral gene transfer of neuronal NOS (nNOS) can decrease central sympathetic outflow, and facilitate cardiac cholinergic transmission, indicating that nNOS confers site-specific actions in relation to its target.

However, a major limitation using viral vectors is the promiscuous nature of transgene expression which can result in the gene of interest being transferred into cells that may not constitutively express nNOS, thereby leading to unwanted effects and confounding the interpretation of the data.

Accordingly, there remains a need for vectors, that are capable of infecting cells with a high efficiency, especially at lower MOIs, and that demonstrate an increased host cell range of infectivity. Provided herein, are methods and compositions that seek to overcome at least some of the aforesaid problems of viral gene therapy.

### SUMMARY OF THE INVENTION

In one embodiment, described herein is a method of inhibiting or suppressing neurotransmission in a nervous system of a subject, comprising the step of causing a innervation in the subject to overexpress nNOS gene, thereby reducing norepinephrine release, causing inhibition or suppression of neurotransmission.

In another embodiment, described herein is a method for producing a viral vector of capable of transferring a nNOS encoding gene into sympathetic nervous system, causing overexpression of nNOS, comprising the steps of: introducing into a selected host cell: a lineraized recombinant shuttle vector comprising: a transcription factors' binding site; followed by a human transcription start site; followed by a nNOS cDNA flanked by a first and second restriction sites; cloned into said first and second restriction sites of a plasmid viral-linker; and a viral backbone; transfecting the lineraized shuttle vector and the viral backbone into the host cells, thereby making a recombinant; digesting the recombinant with a restriction enzyme; transfecting the digested recombinant into an embryonic cell; and recovering the virus.

In one embodiment, described herein is a method of treating pathological conditions arising due to chronic sympathetic activation in a subject, comprising the step of contacting a sympathetic innervation of the subject with a noradrenergic neuron-specific vector resulting in overexpression of nNOS, thereby decreasing neurotransmission.

In another embodiment provided herein is a recombinant shuttle vector comprising: a transcription factors' binding site; followed by a human transcription start site; followed by a nNOS cDNA flanked by a first and second restriction sites; cloned into said first and second restriction sites of a plasmid viral-linker.

In one embodiment, provided herein is a composition comprising a noradrenergic neuron-specific vector.

In another embodiment, provided herein is a method of treating hypertension in a subject, comprising administering to the subject a composition comprising a noradrenergic neuron-specific vector, thereby overexressing nNOS in the subjects cardiac sympathetic nerves.

In one embodiment, provided herein is a method of restoring reduced cardiac vagal activity in a subject, comprising administering to the subject a composition comprising a noradrenergic neuron-specific vector, thereby overexpressing nNOS in the cardiac vagus, increasing nitrous oxide concentration and restoring impaired No-cGMP signaling.

### BRIEF DESCRIPTION OF THE DRAWINGS

FIG. 1. shows: (A) Diagram of Phox2a/Phox2b response site (PRS) in the 5′ proximal area of hDBH gene. PRS×8 promoter contains 8 multimerized PRS sites and a minimum promoter with the TATA box and transcription start site (arrow). (B) pAd.PRS-nNOS shuttle vector map. Following a 240 bp PRS promoter, a 4.3 kb nNOS gene was inserted between SpeI and XhoI site

FIG. 2 shows the difference of eGFP expression observed 3 days post-transduction of Ad.PRS-eGFP (A, C) and Ad.CMV-eGFP (B, D) to cardiac sympathetic neurons isolated from stellate ganglia. Note the transduction of Ad.PRS-eGFP results in exclusive expression of eGFP in neurons as indicated by lack of eGFP expression in any other cells visualized by DAPI. Ad.CMV-eGFP showed widespread eGFP expression in other cells types in neuron culture. Scale bars: 100 μm.

FIG. 3 shows representative fluorescent images of cardiac sympathetic neurons derived from stellate sympathetic ganglia from SD rats 68 h after gene transfer with a noradrenergic neuron-specific vector Ad.PRS-eGFP. (A) GFP expression. (B) Same cell preparation stained with sympathetic neuron marker anti-TH. (C) Overlay of GFP expression and anti-TH stain (Texas-red). Note that all eGFP expressing neurons were colocalized with TH positive neurons. (D) There was no detectable leakage of eGFP expression in other cell types since the cells in the background stained with DAPI did not express eGFP. Scale bar: 50 μm for all the images.

FIG. 4 shows representative fluorescent images of right atria from SD rats 3 days after gene transfer with a noradrenergic neuron-specific vector Ad.PRS-eGFP or a nonspecific adenoviral vector Ad.CMV-eGFP. Pictures A and B are from the same section of atria transduced with Ad.PRS-eGFP. Pictures C and D are from atria transduced with Ad.CMV-eGFP. Note that there was no detectable eGFP expression in intracardiac cholinergic neurons identified by anti-CHAT (Texas-red) as seen in pictures A and B, whereas Ad.CMV-eGFP transduced atria showed widespread transfection in CHAT positive cells and other cells types as seen in pictures C and D. Scale bar: 50 μm for all the images.

FIG. 5 shows (A) Representative Western blot and group data depicting higher nNOS protein abundance in Ad.PRS-nNOS transduced cardiac sympathetic neurons compared with control nontransduced neurons (n=6, *P=0.02). nNOS positive control is nNOS from rat pituitary gland (BD Biosciences). (B) Transduction with Ad.PRS-nNOS on cultured cardiac sympathetic neurons. nNOS was clearly detectable by nNOS antibody (fluorescein, green); nNOS expression in Ad. PRS-nNOS transduced cells colocalized with anti-TH stain (Texas-red). Scale bar: 50 μm for both images. (C) Representative raw data of [3H]NE release following 5 Hz field stimulation in right atrial preparations with (i) Ad.PRS-eGFP, (ii) Ad.PRS-nNOS and (iii) Ad.PRS-nNOS with NOS inhibitor, Nω-Nitro-Larginine (L-NNA) (100 μM). (D) Group data showing Ad.PRS-nNOS treatment significantly decreased (**P<0.01, unpaired t test; n=15) the [3H] NE release compared with Ad.PRS-eGFP control (n=11). L-NNA (100 μM) can reverse this response (**P<0.01, compared with Ad.PRS-nNOS, unpaired t test; n=6).

FIG. 6 shows A: Representative raw data traces showing impaired heart rate responsiveness to right vagal nerve stimulation in the SHR compared to the WKY. B: graph showing significantly impaired responsiveness to 3, 5, and 7 Hz right vagal stimulation in the SHR (n=9) relative to the WKY (n=7; * p<0.05, un-paired t-test).

FIG. 7 shows A, B: Typical data trace showing measurement of [³H]ACh release from isolated atria in response to 5 and 10 Hz field stimulation on time control experiments in WKY (A) and SHR (B). S1 and S2 represent the first and second stimulation respectively. C: [³H]ACh release was significantly impaired at both 5 and 10 Hz field stimulation in the SHR (n=6) compared to the WKY (n=7, **p<0.01 and *p<0.05 respectively, unpaired t-test, response to S1).

FIG. 8 shows A, B: Typical data trace showing effect of the NO donor, SNP (20 μmol/L) on [³H]ACh release response of 5 Hz field stimulation in WKY (A) and SHR (B). C: SNP significantly enhanced the Ach release from the WKY (n=8, †p<0.05, paired t-test), but not in the SHR (n=6). D: showing heart rate response to vagal stimulation in double atrial preparation, SNP significantly increase the bradycardia from the WKY (n=7, †p<0.05, paired t-test), unaffected in the SHR (n=6). (*p<0.05, **p<0.01, ***p<0.001, WKY vs SHR, unpaired ttest)

FIG. 9 shows A, B: Typical data trace showing effect of the sGC inhibitor, ODQ (10 μmol/L) on [³H]ACh release response of 5 Hz field stimulation in WKY (A) and SHR (B). C: ODQ significantly enhanced the Ach release from the WKY (n=7, ††p<0.01, paired t-test), but not in the SHR (n=7). (**p<0.01, WKY vs SHR, unpaired t-test)

FIG. 10 shows A: Raw data trace showing the heart rate response to cumulative additions of CCh (0.1, 0.2 and 0.5 μmol/L) in right double atrial preparations from WKY and SHR. B: 0.1 μmol/L (n=6) and 0.5 μmol/L (n=14) CCh significantly increased heart rate response in SHR compared to WKY (0.1 μmol/L, n=7; 0.5 μmol/L, n=12; *p<0.01, unpaired t-test). No significantly changed was seen in 0.2 μmol/L CCh (WKY, n=14; SHR, n=15).

FIG. 11 shows effects of CCh (0.3 μmol/L) on cGMP efflux concentration in perfused right atrial preparation. No difference in basal cGMP efflux between WKY (n=5) and SHR (n=6). Whereas CCh significantly increased cGMP efflux concentration in the SHR (*p<0.05, unpaired t-test).

FIG. 12 shows NOS activities in atria from Ad.GFP (n=5) and Ad.nNOS (n=5) transfected WKY rats. Activity of nNOS isoform was determined by conversion of [³H]-L-arginine to [³H]-L32 citrulline in the presence of eNOS inhibitor. nNOS activity in Ad.nNOS transfected rats was 23.25±8.44% increase compared to Ad.eGFP transfected rats (*P=0.034, unpaired t test).

FIG. 13 shows Western blot analysis for nNOS and α₁ subunit of guanylate cyclase (α₁₋ₛGC) in the Ad.eGFP (n=5) and Ad.nNOS (n=5) transfected WKY atria. 30 μg of each protein sample was loaded. Top: visualized electrophoresis bands of nNOS, α₁₋ₛGC and β-actin. Bottom: mean data of band densities of nNOS and α₁₋ₛGC normalized by β-actin in Ad.eGFP and Ad.nNOS treated WKY rats (*p<0.05, unpaired t-test). Aorta and forebrain were used to be a positive control for α₁₋ₛGC and nNOS respectively.

FIG. 14 shows A: Heart rate responses to 3-10 Hz right vagal stimulation in Ad.eGFP (n=7, grey bars) and Ad.nNOS (n=5, black bars) transfected SHRs. Vagal responsiveness was significantly enhanced by nNOS gene transfer at all frequencies tested (* p=0.001, unpaired t-test; ** p<0.001, Mann-Whitney Rank Sum test). B: Chronotropic responses of Ad.eGFP (n=22, grey bars) and Ad.nNOS (n=8, black bars) transfected SHR atria to carbachol (0.1 & 0.2 μmol/L). Responses to the muscarinic agonist were unaffected by nNOS gene transfer.

FIG. 15 shows group data comparing the heart rate responses to vagal nerve stimulation in vivo following Ad eGFP and Ad nNOS in the WKY and SHR. Gene transfer of nNOS significantly enhanced the rate response to vagal stimulation in the SHR compared to eGFP treated SHR; 33 augmenting the response to similar levels seen in the WKY treated atria with Ad.eGFP at 3 and 5 Hz.

### DETAILED DESCRIPTION OF THE INVENTION

This invention relates in one embodiment to to gene transfer, specifically, provided herein are methods, vectors and compositions for the transfer of nNOS gene affecting overexpression of nNOS in sympathetic and parasympathetic nervous system and its subsequent use in the treatment of related pathologies.

In one embodiment, autonomic imbalance in central and peripheral nervous system is implicated in the aetiology of hypertension, In another embodiment, similar autonomic imbalance is evident following myocardial infarction (MI) and heart failure. In one embodiment, impaired cardiac parasympathetic regulation and enhanced sympathetic activity characterize cardiac disease states, and are regarded as an independent predictor of mortality. In certain embodiments, bradycardia (e.g. HR<60) and acetylcholine release in response to vagal nerve stimulation is reduced in subjects exhibiting hypertension at the level of the cardiac post-ganglionic neuron. In another embodiment, norepinephrine release from right atria in response to field stimulation in the hypertensive subjects, is higher compared to the non-hypertensive subjects. This gives direct evidence that the sympathetic nervous system is hyper-reactive in hypertensive subjects at the level of heart.

In one embodiment, the term “hypertensive”, or “hypertensive subject” refers to a subject exhibiting symptoms associated with hypertensive disease. In one embodiment, such hypertensive disease is pulmonary hypertensive diseases, which comprises all conditions characterized by an increase in the blood pressure within the blood vessels supplying the lungs thereby capabale of increasing the complications associated with pulmonary embolism, heart failure, valvular disease, chronic lung diseases and autoimmunity. In another embodiment, hypertensive disease refers to diseases and pathological conditions relating to or involving the heart or blood vessels. Examples of cardiovascular diseases include all forms of ischemic heart disease, cardiac dysrhythmia and cardiac arrhythmia, congestive heart failure, and hypertensive disease listed in International Classification of Diseases, Vol. 9, Clinical Modification, Easy Coder (1997) (“ICD 9 CM”) (incorporated herein by reference in its entirety for all purposes), as well as all cardiovascular diseases responsive or sensitive to vasodilation, and all cardiovascular diseases described in E. Braunwald, HEART DISEASE: A TEXTBOOK OF CARDIOVASCULAR MEDICINE (3d ed. 1988) (incorporated herein by reference in its entirety for all purposes).

In one embodiment, overexpression of the nNOS gene in the parasympathetic nervous system of the subject, affected through the methods, vectors and compositions described herein, causes increase in nNOS activity. Transduction with Ad.PRS-nNOS increases in one embodiment, the nNOS activity in the atrial extracts, and in another embodiment, enhances atrial cGMP levels.

In one embodiment, the sympathetic innervation in which nNOS activity is increased using the vectors, methods and compositions described herein, is cardiac sympathetic neurons. In another embodiment, causing a sympathetic innervation of the subject to overexpress nNOS gene is effected by a viral vector, which, in certain embodiments is an adenoviral vector, lentiviral vector, a retroviral vector, an adeno-associated viral vector, or a combination thereof

“Transduction” refers in one embodiment to the transfer of genetic material or characteristics from a host cell to a target cell by a DNA construct, such as bacteriophage in one embodiment, or plasmid in another embodiment.

In one embodiment, essential hypertension is neurogenic, with high rates of spillover of norepinephrine (NE) from the heart and kidneys. The increased cardiac and renal spillover of NE is attributable in another embodiment, to increased sympathetic nerve firing rates, or activation of sympathetic efferents in another embodiment of sympathetic outflow, to the skeletal muscle vasculature.

In one embodiment, gene transfer using viral vectors expressing nitric oxide synthases (NOS) enhance the signaling role of nitric oxide (NO) in regulating endothelial function, ventricular myocyte calcium handling and autonomic neurotransmission. Viral gene transfer of neuronal NOS (nNOS) decreases in another embodiment, the central sympathetic outflow, and facilitate cardiac cholinergic transmission, indicating that nNOS confers site-specific actions in relation to its target.

According to this aspect of the invention, and in one embodiment, provided herein is a method of inhibiting or suppressing neurotransmission in a nervous system of a subject, comprising the step of causing a innervation in the subject to overexpress nNOS gene, thereby reducing norepinephrine release, causing inhibition or suppression of neurotransmission.

In one embodiment, Nitric oxide (NO), is a ubiquitous signaling messenger molecule involved in diverse pathophysiologic processes such as neurotransmission, inflammatory and immune responses, and vascular homeostasis. NO is not stored once produced; and diffuses freely to its site of action where in one embodiment, it binds covalently to its effectors.

NO is synthesized in one embodiment by the action of a group of enzymes called NOSs which convert the amino acid L-arginine into NO and another amino acid, L-citrulline. NOSs contain four cofactors: FAD, FMN, tetrahydrobiopterin, and haem; the haem center has spectral properties resembling those of cytochrome P₄₅₀. There are three types of NOSs. Two are constitutive (named cNOS) and one that is inducible by cytokines and endotoxins (named iNOS). There are two subtypes of cNOS: one in the vascular endothelium named eNOS and the other is present in the central and peripheral nervous systems named NNOS. nNOS and eNOS are Ca²⁺/calmodulin-dependent enzymes. nNOS is found in a variety of neurons in both the central and peripheral nervous systems and is a constitutionally expressed enzyme. In certain embodiments, it can be induced in neurons by certain treatments.

In one embodiment, the signaling pathway responsible for nNOS-derived NO inhibiting sympathetic neurotransmission involve NO modifying cell physiology through activation of soluble guanylyl cyclase (sGC) and subsequent induction of cGMP production, which in turn activates cGMP-dependent protein kinase and phosphodiesterases that decrease cAMP-dependent phosphorylation of neuronal Ca²⁺ channels. In another embodiment, sGC, is the main target protein for NO and is markedly desensitized or down-regulated in hypertension.

In one embodiment, down regulation of the α₁ subunit of sGC in the atria and aorta of the hypertensive subjects, compared to non-hypertensive subjects. In another embodiments, guanylate cyclase inhibition increased norepinephrine (NE) release in non-hypertensive subjects, but is non-effective in the hypertensive subjects, suggesting functional uncoupling of NO to its second messenger. In one embodiment, tissue levels of cGMP are between about 15 to about 25% lower in the hypertensive subjects compared to the non-hypertensive subjects. Conversely, and in another embodiment, decreased sGC expression and cGMP production accounts in one embodiment for impaired vasodilatation in the Africo-Carribean population, whom are susceptible to hypertension, or fetal programming of hypertension, and in pulmonary hypertension in other embodiments. In one embodiment, the methods, vectors and compositions described herein, may be used in the treatment of the above-mentioned pathologies.

Likewise and in one embodiment, down-regulation of components of the sGC-dependent pathway in hypertensive subjects. Superoxide production trigger in one embodiment the desensitization of vascular sGC in hypertension. In one embodiment, levels of tetrahydrobiopterin and total biopterin are normal in atria from the hypertensive subjects; and in another embodiment, nNOS activity in the hypertensive subjects remains unaltered, indicating that uncoupling of nNOS from its main co-factor is not involved in the mechanism of peripheral sympathetic dysfunction in the hypertensive subject.

According to this aspect of the invention; and in one embodiment, provided herein is a method of inhibiting or suppressing neurotransmission in the parasympathetic nervous system of a subject, comprising the step of causing a innervation in the subject to overexpress nNOS gene, thereby reducing norepinephrine release, causing inhibition or suppression of neurotransmission.

The autonomic nervous system includes sympathetic and parasympathetic pathways. In one embodiment, parasympathetic nerves activation causes a decrease in atrial rate and contractile force, atrio-ventricular nodal conduction, and ventricular contractile force.

In one embodiment, the replication-deficient adenoviral vectors (referring in one embodiment to a virus that cannot replicate in a host cell), used in the methods and compositions described herein, and which encode recombinant nNOS under control of the noradrenergic neuron-specific promoter, attenuate the sympathetic hyper-responsiveness in the hypertensive subjects. The noradrenergic specificity of Ad.PRS-nNOS transduction is confirmed in certain embodiments, by co-localization of tyrosine hydroxylase positive neurons with nNOS This promoter is highly specific with no detectable leakage of virus into other cell types.

The AAV2 genomic plasmid pTR is modified in one embodiment, by replacing the 1.8-kb NSE promoter with human dopamine ?-hydroxilase (hDBH) promoter immediately upstream of the human nNOS cDNA. In another embodiment, the NSE promoter is retained unmodified. NSE refers in another embodiment to neuron-specific enolase (NSE) promoter, which directs panneuronal expression of fusion gene constructs in the CNS of the target subject. In one embodiment, the shuttle vector used in the compositions, vectors and methods described herein is AAV2-NSE-nNOS, AAV2-hDBH-nNOS, or a combination thereof. In one embodiment, the vector used in the compositions and methods described herein, is Ad-pTR-nNOS, Ad-PRS-nNOS or a combination thereof.

The term “adenovirus” or “adenoviral” (of the adenovirus), refers in one embodiment to a family of icosahedral (20-sided) viruses that contain DNA. Two genuses, Mastadenovirus and Aviadenovirus are included in the adenovirus family. While there are over 40 serotype strains of adenovirus, most of which cause benign respiratory tract infections in humans, subgroup C serotypes 2 or 5 are predominantly used as vectors. The life cycle does not normally involve integration into the host genome, rather an adenovirus replicates as episomal elements in the nucleus of the host cell and does not insert into the genome. In one embodiment, “adenoviral vector” refers to a vector derived from publicly available adenoviral DNA. In another embodiment, an adenoviral vector includes the inverted terminal repetitions of an adenovirus. In other embodiments, vectors used in the methods and compositions described herein, can include elements from other viruses, such as retroviruses.

The vectors which are introduced into the tissues or organs are taken up by the synaptic regions of these tissue or organ-associated neurons. The term, “taken up” refers in one embodiment, to either a passive or an active mechanism for moving the vectors into the the neuron. Examples of such mechanisms are receptor mediated processes, endocytosis and vesicle mediated processes. Once present in the cell body of the neuron, the nNOS-encoding genes delivered by the vector described herein, are transported into the nucleus where they are found to be expressed by the neuron.

Viral vectors, such as adenoviral in one embodiment, or retroviral vectors, in other embodiments, are used to introduce foreign DNA with high efficiency into target cells. The wild type adenovirus genome is approximately 36 kb, of which up to 30 kb can be replaced with foreign DNA. There are four early transcriptional units (E1, E2, E3 and E4), which have regulatory functions, and a late transcript, which codes for structural proteins. Replication-defective vectors are produced, which in one embodiment have an essential region of the virus (e.g. E1,) deleted. Other genes (e.g. E3 or E4) can be also deleted in other embodiments, in the replication-deficient vectors. These additional gene deletions increase the capacity of the vector to carry exogenous nucleic acid sequences. In another embodiment, the E2 region can also be deleted in a replication-defective vector; this type of vector is known as a “mini Ad,” “gutted vector,” or “gutless vector.” In one embodiment, to utilize these “gutless vectors”, a helper cell line, (e.g. AD-293, HEK293 cells), is needed to provide necessary proteins for virus packaging. In one embodiment, the viral vector used to introduce the nNOS-encoding gene into the cardiac sympathetic neurons, is an adenovirus.

Adenoviral packaging cell lines arc cells including nucleic acid molecules that encode adenoviral capsid proteins which can be used to form adenoviral particles. The adenoviral particles are competent to package target adenovirus which has a packaging site

In another embodiment, retroviruses, such as Moloney murine leukemia virus (MoMLV), are used to introduce the nNOS-encoding gene into the cardiac sympathetic neurons.

Retroviruses are RNA viruses that, which when infecting cells, convert their RNA into a DNA form that is then integrated into the cellular genome. In one embodiment, the integrated provirus produces RNA from a promoter located in the long terminal repeats (LTRs), which are DNA repeats located at the end of the integrated genome. Retroviral DNA vectors are plasmid DNAs which contain two retroviral LTRs, and a gene of interest, such as the gene encoding NNOS in one embodiment, inserted in the region internal to these LTRs. In one embodiment, the retroviral vector is packaged by packaging cell lines, containing the gag, pol, and env genes, which provide all the viral proteins required for capsid production and the virion maturation of the vector. A retroviral vector integrates into the cellular genome once it is introduced into cells, thereby stably transfecting the cells.

Retrovirus refers in one embodiment to any virus in the family Retroviridae. These viruses have similar characteristics, specifically they share a replicative strategy. This strategy includes as essential steps reverse transcription of the virion RNA into linear double-stranded DNA, and the subsequent integration of this DNA into the genome of the cell. All native retroviruses contain three major coding domains with information for virion proteins: gag, pol and env. In one embodiment, a retrovirus is an avian sarcoma and leukosis virus, a mammalian B-type virus, a Murine leukemia-related virus, a Human T-cell leukemia-bovine leukemia virus, a D-type virus, a lentivirus, or a spumavirus. In another embodiment, the virus is a Rous sarcoma virus, a mouse mammary tumor virus, a human T-cell leukemia virus, a Mason-Pzifer monkey virus, a human immunodeficiency virus, a human foamy virus, or a Molony Leukemia Virus. In other embodiments, a retrovirus contains three genes known as “gag,” “pol,” and “env.”

In one embodiment, provided herein is a method for producing a viral vector capable of transferring a nNOS encoding gene into sympathetic nervous system, causing overexpression of nNOS, comprising the steps of: introducing into a selected host cell: a lineraized recombinant shuttle vector comprising: a transcription factors' Phox 2a/2b binding site; followed by a human transcription start site; followed by a nNOS cDNA flanked by a first and second restriction sites; cloned into said first and second restriction sites of a plasmid viral-linker; and a viral backbone; transfecting the lineraized shuttle vector and the viral backbone into the host cells, thereby making a recombinant; digesting the recombinant with a restriction enzyme; transfecting the digested recombinant into an embryonic cell; and recovering the virus.

The term “Vector” refers in one embodiment to a nucleic acid molecule as introduced into a host cell, thereby producing a transformed host cell. In one embodiment, a vector may include nucleic acid sequences that permit it to replicate in the host cell, such as an origin of replication. In another embodiment, a vector includes sequences encoding one or more therapeutic genes and/or selectable marker genes and other genetic elements known in the art. A vector can transduce in one embodiment, or transform or infect a cell in another embodiment, thereby causing the cell to express or over express nucleic acids and/or proteins other than those native or normative to the cell. A vector includes in certain embodiments, materials to aid in achieving entry of the nucleic acid into the cell, such as a viral particle, liposome, protein coating or the like. A vector may be a viral vector, derived from a virus, such as an adenoviral vector in some embodiments.

The terms “Transduced”, “Transfected”, and “Transformed” refer in one embodiment to the ability of a virus or vector to “transduce” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication. In another embodiment, the term transformation or “transduction” encompasses all techniques by which a nucleic acid molecule might be introduced into such a cell, including transfection with viral vectors, transformation with plasmid vectors, and introduction of naked DNA by electroporation, lipofection, and particle gun acceleration. In one embodiment, cardiac sympathetic neurons are transduced by the vectors described herein and thereby are transformed or transfected to overexpress nNOS-encoding gene, resulting in overexpression of nNOS in the transfected neurons.

The term “shuttle vector” refers in one embodiment to a vector which shuttles a gene, but does not include all of the components necessary for production of a viable virion.

In one embodiment, stimulus-induced phosphorylation of p47^{phox} disrupts the intramolecular interaction to render the Phox domain in a state accessible to phosphoinositides, which promotes membrane translocation of this protein, playing a crucial role in activation of the phagocyte NADPH oxidase. In another embodiment, the transcription factors' Phox 2a/2b binding site in the vectors used in the compositions and methods described herein, facilitate the transduction of the shuttle vector into the cell.

In one embodiment, the linearized recombinant shuttle vector used in the methods, vectors and compositions described herein, further comprises at least one tandem repeat of the transcription factors' Phox 2a/2b binding site, or two tandem repeat of the transcription factors' Phox 2a/2b binding site, or three tandem repeats of the transcription factors' Phox 2a/2b binding site, or four tandem repeats of the transcription factors' Phox 2a/2b binding site, or five tandem repeats of the transcription factors' Phox 2a/2b binding site, or six tandem repeats of the transcription factors' Phox 2a/2b binding site, or seven tandem repeats of the transcription factors' Phox 2a/2b binding site, or eight tandem repeats of the transcription factors' Phox 2a/2b binding site, or n tandem repeats of the transcription factors' Phox 2a/2b binding site in other embodiments.

In another embodiment, the selected host cell into which the shuttle vector is introduced, for making the vector used in the compositions and methods described herein, is a BJ5183 competent cell. In one embodiment, the term “competent cell” refers to one internally carrying all the functions necessary for complementation of the defective virus. These functions are preferably integrated in the genome of the cell, thereby reducing the risks of recombination and endowing the cell line with enhanced stability

The method provided herein and is based in one embodiment on the use of a competent cell, such as BJ5183, to provide the complementing functions. In one embodiment, the competent cells do not express any function of transduction of the defective recombinant genome used in the linearized shuttle vector. In this case, it is possible to use either a viral vector comprising all the functions necessary for the transduction of the defective recombinant genome, or several viral vectors each carrying one or more of the functions necessary for the transduction of the defective recombinant genome. It is also possible to use a population of competent cells capable of already transducing one or more functions of the defective recombinant genome (encapsidation line). In this case, the viruses used will provide only the functions necessary for the transduction of the defective recombinant genome which are not already transduced by the competent cells.

In one embodiment, the vectors described herein include an upstream promoter for controlling transcription of the cDNA of NOS coding sequence. In another embodiment, transcriptional repressors are also used. The promoter is a constitutive promoter in one embodiment, or a regulated or inducible promoter in other embodiments. In one embodiment, the promoter is not the promoter with which the NOS coding sequence is associated in nature i. e. the nucleic acid is a heterologous construct. In another embodiment, the promoter is naturally-occurring, albeit a chimeric regulatable system incorporating various prokaryotic and/or eukaryotic elements. Various promoter modules are used in certain embodiments, to allow various levels of control. Constitutive promoters useful for directing transcription of the NOS coding sequence include those from genes coding for glycolytic enzymes in one embodiment, or from p-actin, and allow persistent up-regulation of NOS expression in other embodiment. In one embodiment, viral promoters are used e. g. from CMV in another embodiment.

In one embodiment, tissue-specific or cell-type-specific promoters used in the vectors described herein, which are used in the compositions and methods described herein, facilitate spatial control. In another embodiment, provided herein is a promoter which is active in neuronal tissue, particularly tissue in the autonomic nervous system (e. g. the vagus). In one embodiment, promoters are active in cholinergic ganglia tissue e. g. the promoter from choline acetyltransferase or from the vesicular acetylcholine transporter in another embodiment. In one embodiment, the promoters used in the shuttle vectors, viral vectors or compositions described herein—is a Drug-inducible promoter. Drug-inducible promoters facilitate temporal control in another embodiment, by including cAMP response element enhancers in a promoter, therefore in another embodiment, cAMP modulating drugs such as such as prostaglandin E (PGE) are used. A repressor elements is included in an embodiment of a vector to prevent transcription in a drug's presence.

Transfer and recombination of DNA segments, including coding sequences and non-coding sequences, is carried out in one embodiment using restriction endonuclease enzymes. Restriction endonucleases are especially useful because each one introduces a hydrolytic cleavage of a phophodiester bond linking adjacent nucleotides of a DNA structure only at a specific, defined site. The site of cleavage is defined by a sequence of nucleotides surrounding or adjacent to the cleavage site. Over one hundred such enzymes are now known, and the sites at which they act, referred to in one embodiment as “restriction sites”, are defined. In one embodiment, the nucleotide sequence defining the restriction site and the bonds cleaved is specific for each enzyme. In other embodiments, different enzymes recognize the same site or a variant, such as the same sequence with a methylated base, or a shorter subsequence, and act to hydrolyze the same bond, or different bonds within or adjacent to the recognition sequence. Some restriction enzymes hydrolyze bonds adjacent to complementary bases on double-stranded DNA, producing “blunt ends.” Others produce staggered cuts which result in the DNA having overlapping complementary or “sticky” ends.

In one embodiment, the region essential to viral viability lies in or in proximity to the deletion site. In another embodiment, other insertion sites are used, such as, for example, restriction sites already present in the host cell wild-type genome. In one embodiment, additional modifications in the recombinant viral genome are made to facilitate manipulations, such as the insertion of unique restriction sites in various intergenic regions (e.g., a unique SpeI, or XhoI site flanking the nNOS encoding gene).

In one embodiment, the either the first or second restriction site used in the method of producing the viral vector described herein, is SpeI, or XhoI. In one embodiment, the “Shuttle” vector as used herein refers to a vector designed to serve as a carrier of a particular DNA segment from one vector to another. In another embodiment, a shuttle vector is used for combining particular DNA elements. For example, a lineraized shuttle vector as described herein, is provided with a cloning site with at least two restriction sites flanking at least one other restriction site. The two rare-cutter sites may be the same or different. By inserting a particular desired DNA segment into the vector at a middle site, then excising with the appropriate rare-cutting endonuclease, the desired DNA can be isolated with rare-cutter site sequence at either end. The desired DNA can then be readily inserted into any vector provided with the same rare cutting endonuclease site.

In one embodiment, the human embryonic cells into which the digested recombinant DNA is transfected and which are used in the methods and compositions described herein, are helper cells as described hereinabove, such as AD-293 in one embodiment, or HEK293 cells or a combination thereof in other embodiments.

In one embodiment, the human transcription start site used in the vectors described herein, for use in the methods and compositions described herein, is human dopamine ?-hydroxilase (hDBH) promoter. In one embodiment, the associated transcriptional elements that are used as promoter systems, are implemented to restrict suicide gene expression to the neuronal cells, such as the promoter fragments of the genes for neuron-specific dopamine-β-hydroxylase (DBHp).

In another embodiment, dopamine-β-hydroxylase (DBHp) catalyzes the conversion of dopamine to noradrenaline, the third step of catecholamine biosynthesis. It is localized in noradrenergic and adrenergic neurons of central nervous system, sympathetic ganglia, and adrenal medulla chromaffin cells. In one embodiment, the 5′ region of the DBH gene contains multiple sequence elements involved in both positive and negative control of DBH gene expression. The DBH1.1 nlacZ gene directs 3-galactosidase expression to adrenal chromaffin cells, or central and peripheral noradrenergic neurons, and other neurons in which DBH is detected in other embodiments, such as cranial parasympathetic and enteric neurons. This length of DBH promoter sequence is sufficient in another embodiment to direct fetal expression of ?-galactosidase to noradrenergic neurons. In another embodiment, sequence elements essential for expression in noradrenergic neurons are present between −0.6 kb and −1.1 kb relative to the human DBH transcriptional start site. In one embodiment, choosing the right neuron specific promoter may be used to direct the overexpression of nNOS using the vectors in the compositions and methods described herein.

In one embodiment, the restriction enzyme used to prepare the vector in the methods described herein, for the methods and compositions described herein, is PacI.

In another embodiment, the viral linker used to operably link the DNA construct to the viral backbone is pAd-Transcription Factor Binding Site-Linker, such as pAd-PRS-Linker, or in another embodiment, pTR-Linker. In one embodiment, the cloning site is produced by ligating nucleotide sequences (linkers) onto the DNA termini; these ligated linkers may comprise in other embodiments, specific chemically synthesized oligonucleotides encoding restriction endonuclease recognition sequences. In one embodiment, the linkers used in the vectors described herein Enforce the interaction between the DNA construct (e.g. plasmid containing the cDNA of the gene encoding nNOS) and the viral backbone, by inserting various lengths of oligonucleotide or propanediol phosphate linkers at the opposite strand of the components described herein.

In another embodiment, the methods provided herein, further comprise the step of isolating the recombinant virus by plaque assay, thereby creating a primary viral stock. In another embodiment, the primary viral stocks is amplified in Ad-293 cells until a cytopathic effect is observed, prior to recovering the virus.

In one embodiment, the vectors described hereinabove are used in the methods described herein. Accordingly, provided herein is a method of treating pathological conditions arising due to chronic sympathetic activation in a subject, comprising the step of contacting a sympathetic innervation of the subject with a noradrenergic neuron-specific vector resulting in overexpression of nNOS, thereby decreasing neurotransmission. In one embodiment, the vectors described hereinabove, are the neuron-specific vectors used in the methods described herein, used for the treatment of pathological conditions arising due to chronic sympathetic activation in a subject.

The sympathetic nervous system (SNS) is responsible for the central nervous system (CNS) ability in maintaining homeostasis. CNS-mediated SNS activation is produced in one embodiment, in response to short-term changes in the physiological state, or in another embodiment in response to chronic pathophysiological disorders such as congestive heart failure, or essential hypertension and the like. In another embodiment, prolonged stimulation of the SNS depletes NE stores rapidly and leads to an increase in the neuronal release of dopamine, adenosine triphosphate (ATP), adenosine, and prostaglandins.

In one embodiment, under circumstances of chronic activation of the SNS, the plasma concentration of the sympathetic neurotransmitter norepinephrine (NE) is increased. The plasma concentration of NE, is determined in one embodiment, by the rates of release of NE to plasma and removal of NE from plasma. In one embodiment, plasma NE clearance is reduced, and the plasma concentration thereby increases, because of the reduced cardiac output and organ blood flows that accompany congestive heart failure. In one embodiment, contacting the SNS with vectors described herein, will reduce the evoked release of NE into the plasma, thereby treating congestive heart failure. In another embodiment, contacting the SNS with the vectors described herein, using the methods described herein, further comprises contacting the SNS with sodium nitroprusside.

In one embodiment, Heart failure (HF) is a complex disorder leading to a disturbance of the normal pumping of blood to the peripheral organs thereby meeting the oxygen demands of the body as it responds to its environment. In another embodiment, HF eventually occurs in a heart that has suffered myocardial damage, regardless of the initial cause of the damage (hypertension, myocardial ischemia, cardiomyopathy, etc.), if such damage persists for a prolonged period. In one embodiment, compensation for the myocardial damage and maintenance of hemodynamics occurs via the chronic activation of the sympathetic nervous system, resulting in LV dilatation and/or hypertrophy.

In one embodiment, the malignancy associated with chronic SNS activation, sought to be treated using the methods, vectors and compositions described herein, is heart failure, hypertension, sudden cardiac death, myocardial infarct or a combination thereof. In another embodiment, the overexpression of nNOS in the sympathetic innervation of the subject resulting from the administration of the compositions or vectors described herein and using the methods described herein, reduces ?-adrenergic stimulation while maintaining the regulation of sympathetic discharge, thereby meeting cardiac output in response to the subject's activity.

Beta-adrenergic regulation of cardiac contraction is coupled in certain embodiments to elevations in adenosine (cAMP) and guanosine (cGMP) cyclic nucleotides. In another embodiment, increased plasma cAMP concentration enhances cardiac contractility by activating protein kinase A (PKA), whereas contemporaneous stimulation of cGMP opposes this in another embodiment, by activating protein kinase G (PKG-1). The stimulation of cGMP is attributable in one embodiment to stimulation of soluble guanylate cyclase (sGC) by NO. Cyclic GMP is also synthesized by receptor GC (rGC) coupled to natriuretic peptide stimulation, and both sources can modulate cardiac function and structure, particularly in hearts chronically stimulated by neurohonnones such as NE in one embodiment or mechanical stress. In one embodiment, cardiodepression due to the NO-cGMP pathway has pathophysiologic significance because its activity is increased with heart failure.

In one embodiment, nNOS is expressed in orthosympathetic nerve terminals and regulates the release of catecholamines in the heart.

In one embodiment, the viral vectors described hereinabove, are interchangeable with the suttle vectors described herein, and are used in the vectors and compositions described herein. In another embodiment, provided herein is a recombinant shuttle vector comprising: a transcription factors' Phox 2a/2b binding site; followed by a human transcription start site; followed by a nNOS cDNA flanked by a first and second restriction sites; cloned into said first and second restriction sites of a plasmid viral-linker. In one embodiment, the shuttle vector is a viral vector.

In another embodiment, the shuttle vector described herein, further comprising at least one tandem repeat of the transcription factors' Phox 2a/2b binding site. In another embodiment, the shuttle vector described herein, further comprising one tandem repeat of the transcription factors' Phox 2a/2b binding site, or two tandem repeats of the transcription factors' Phox 2a/2b binding site, or three tandem repeats of the transcription factors' Phox 2a/2b binding site, or four tandem repeats of the transcription factors' Phox 2a/2b binding site, or five tandem repeats of the transcription factors' Phox 2a/2b binding site, or six tandem repeats of the transcription factors' Phox 2a/2b binding site, or seven tandem repeats of the transcription factors' Phox 2a/2b binding site, or eight tandem repeats of the transcription factors' Phox 2a/2b binding site, or n tandem repeats of the transcription factors' Phox 2a/2b binding site.

In one embodiment, the vectors described hereinabove, are used in the methods described herein. Accordingly and in one embodiment, provided herein is a method of treating hypertension in a subject, comprising administering to the subject a composition comprising a noradrenergic neuron-specific vector, whereby the vector is a viral vector, said vector comprises a nucleic acid construct comprising a gene encoding nNOS flanked by a first and a second restriction sites whose expression is controlled by a first and a second promoter, such that said gene is expressed in sympathetic nerves and overexpresses nNOS, thereby overexressing nNOS in the subjects cardiac sympathetic nerves.

In one embodiment, oxidative stress impairs the nitric oxide (NO)—cGMP pathway, resulting in diminished contractile response of the cardiac muscle resulting from certain pathophysiological events, such as chronic SNS activation, essential hypertension ischemia-reperfusion injury, myocardial infarct and the like.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combination thereof in other embodiments.

“Treating” embraces in another embodiment, treating is inhibiting, ameliorating, reducing blood pressure or a combination thereof. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term. A method to treat hypertension may comprise in one embodiment, a method to increase expression of nNOS in chronically activated cardiac SNS, since the latter may lead to, or aggravate hypertension.

In another embodiment, provided herein is a method of restoring reduced cardiac vagal activity in a subject, comprising administering to the subject a composition comprising a noradrenergic neuron-specific vector, whereby the vector is a viral vector, said vector comprises a nucleic acid construct comprising a gene encoding nNOS flanked by a first and a second restriction sites whose expression is controlled by a first and a second promoter, such that said gene is expressed in sympathetic nerves and overexpresses nNOS in the cardiac vagus, increasing nitrous oxide concentration and restoring impaired NO-cGMP signaling.

In another embodiment, reduced cardiac vagal activity results in reduced respiratory sinus arrhythmia, low RR interval variability, and low baroreflex sensitivity. In one embodiment, a significant component of parasympathetic dysfunction occurs peripherally within the efferent cardiac vagal neurons of hypertensive subjects. Accordingly and in one embodiment, reduced cardiac vagal activity, which is treated using the methods described herein may result in hypertension of the subject unless treated.

In one embodiment, the methods for the treatment of hypertension resulting from reduced cardiac vagal activity, comprising the administration to the subject of a composition comprising a noradrenergic neuron-specific vector, whereby the vector is a viral vector or a shuttle vector in another embodiment, said vector comprises a nucleic acid construct comprising a gene encoding nNOS flanked by a first and a second restriction sites whose expression is controlled by a first and a second promoter, such that said gene is expressed in parasympathetic nerves such as the cardiac vagal neiurons and overexpresses nNOS, further comprises administering to the subject an effective amount of sodium nitroprusside (SNP), thereby increasing supply source for NO, or in another embodiment, soluble guanylate cyclase (sGC), thereby restoring impaired NO-cGMP signaling.

In one embodiment, the compositions described herein, which, in another embodiment are used in the methods described herein, further comprise sodium nitroprusside, soluble guanylate cyclase, or their combination.

Heart rate (HR) is modified in one embodiment by the autonomic nervous system as determined by the balance between sympathetic influences, which increase HR and parasympathetic discharges, which cause deceleration of HR, both affecting the intrinsic rate of spontaneous depolarization of the sinoatrial (SA) node. At rest there is a tonic level of activity in each of these components and heart rate depends on the interplay between the sympathetic and parasympathetic influences whereby parasympathetic (vagal) tone predominates. In another embodiment, the relationship between R-R wave interval and the frequency of cardiac vagal stimulation is linear, yielding an incremental activity in vagal efferents, which prolongs the R-R interval by a fixed value independent of the initial R-R interval.

In another embodiment, the term “restoring” refers to increase in the release of acetylcholine and enhancing heart rate (HR) in response to vagal nerve stimulation (VNS). In another embodiment, reduced cardiac vagal activity in patients with myocardial infarction (MI) is associated with a high risk of sudden death. Accordingly and in one embodiment, provided herein is a method of reducing risk of sudden death in patients with MI, comprising administering to the patients the compositions described herein. In another embodiment, overexpression of nNOS in cardiac vagus of patient with MI is effected using the methods described herein.

In one embodiment, vagal impairment in a hypertensive subject resides within the peripheral nervous system at the level of NO-sGC-cGMP pathway. In another embodiment, administration of a NO donor (e.g. SNP) produces a pre-junctional enhancement of cholinergic neurotransmission in the right atria of the normotensive subject and is absent in the atrial tissue a hypertensive subject. Therefore, in one embodiment a relatively high local concentration of NO is required to increase the bioavailability of NO under conditions of increased oxidative stress present in hypertension, since in the presence of the superoxide anion NO will undergo rapid conversion to peroxynitrite (ONOO—), thereby inhibiting in one embodiment, release of Acetylcholine. In another embodiment, nNOS gene transfer using the vectors and compositions described herein, with the methods described herein, provides a more potent NO signal than the signal attained with a NO donor alone, upregulating sCG in hypertensive subjects as in the a normotensive subject. Accordingly and in one embodiment, described herein is a method of upregulating sGC expression in a hypertensive subject, comprising the step of administering to the subject the compositions described herein, thereby overexpressing nNOS within vagal neurons of the subject.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

Construction of Noradrenergic Neuron-Specific Adenoviral Vector

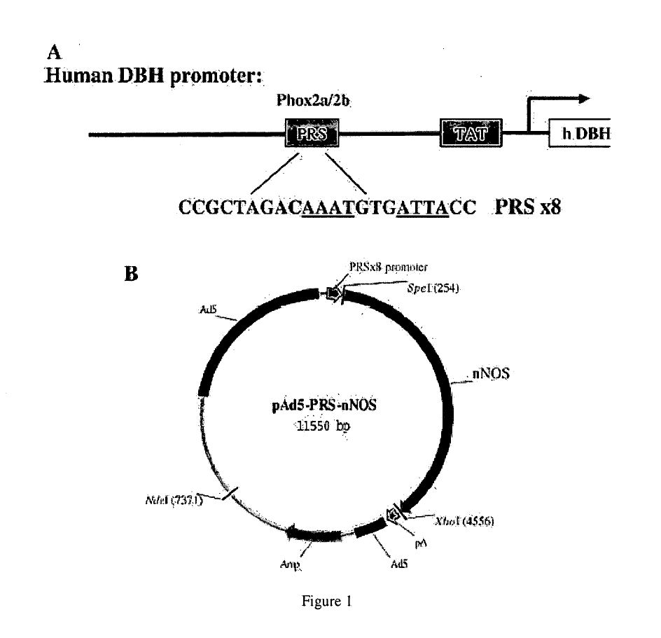

The PRS×8 promoter consists of an eight tandem repeat of transcription factors Phox 2a/2b binding site, followed by a transcription start site from human dopamine P-hydroxylase (hDBH) promoter. The total length of PRS×8 promoter is 240 bp [10]. pAd-PRS×8-nNOS was constructed by inserting nNOS cDNA flanked by restriction sites SpeI and XhoI. The resulting PCR fragment was cloned into the SpeI and XhoI site of pAd-PRS-linker vector (FIG. 1).

Adenovirus Production and Concentration

Recombinant adenoviral plasmid was produced by transfection of the linearized shuttle vector together with the adenoviral backbone into BJ5183 competent cells. The recombinant was digested with PacI to expose its inverted terminal repeats (ITR) and then was transfected to AD-293 cells [11]. Recombinant virus was isolated by plaque assay. Primary viral stocks were amplified in Ad-293 cells until a cytopathic effect was observed. Cells were then harvested and lysed to release the virus. Pure virus was recovered from the lysate using Adenopure Kit (Puresyn, Inc.). The viral particle: plaque forming unit (vp/pfu) ratio for Ad.CMV-eGFP is 15.1, for Ad.PRS-eGFP and Ad.PRSnNOS is 20:1.

Cardiac Sympathetic Neuron Isolation and Transduction

Neonatal SD rats aged 4-10 days were sacrificed by exsanguination while under halothane anesthesia. Stellate ganglia were isolated and transferred into PBS, pH 7.4. Tissue and adipose were removed and the ganglia were cut into smaller pieces. Neurons were isolated by triturating in a mixture of collagenase Type I (130 U/ml, Worthington) and trypsin (1 U/ml, Sigma) followed by 30 min incubation at 37° C. with 5% CO2. Fibroblast contaminations were removed by a panning step, where cells were incubated on a sterile glass cover slip (22 mm) for 2 h with 5% CO2 at 37° C. Neurons were then carefully collected and cultured in 4 well dishes (Culture area, 1.9 cm2/well, Nunc, Denmark) coated with poly-L-lysine (0.01%, Sigma) and laminin (40 μg/ml, Invitrogen). Culture medium was renewed every 2 days.

Gene Transfer to Freshly Isolated Neonatal Rat Atria and Aged Rat Heart

Right atria from neonatal rats were removed and transferred to 4 well culture dishes (Culture area, 1.9 cm2/well, Nunc, Denmark) with growth medium (Dulbecco's modified eagle's medium (DMEM), 4500 mg/l glucose, 10% fetal bovine serum (FBS), 2 mM L-glutamine. The cells were then infected with 4−8×10⁸ pfu of viral vectors per well. The virus-containing medium was left in the well no longer than 12 h before changing to fresh medium. For aged heart atrial injection, 12-16 week old SD rats were anesthetized under 4% isofluorane in oxygen at 4 l per minute. After the deep reflexes were absent, 3×10⁹ pfu of viral vector in phosphatebuffered saline was injected into the right atrium via the right 4th intercostal space. The rats were allowed to recover and were monitored for 4 h before being transferred to the animal care facility where they were monitored daily until terminal procedure.

Immunohistochemistry

Cultured primary neurons were fixed with 4% Paraformaldehyde and permeabilized with 0.1% Triton X100 and 1% BSA. Cells were then processed for immunoreactivity with mouse anti-tyrosine hydroxylase (TH), 1:200 (Sigma), goat anti choline acetyltransferase (CHAT) or rabbit anti mouse nNOS, 1:200 (Zymed). Fixed cells were incubated sequentially with 10% normal horse serum, primary antibody and biotinylated horse anti-mouse (rat adsorbed), anti goat or anti rabbit IgG 1:200 respectively. Then the cells were incubated with Streptavatin Texas red, or Streptavatin fluorescein 1:200.

Fluorescent Imaging and Assessment of Colocalization

Images of living GFP expressing neurons and the fixed cells were obtained using a inverted fluorescent microscope (Nikon TE2000U) with a cooled CCD color camera (DS5Mc, Kodak). More than 10 viral transduction experiments and over 10 fields of view for each experiment were evaluated. Cells showing GFP colocalization with TH were determined using color overlay and merging on individual images in the same field using imaging software Metatmorph, Molecular Devices, U.S.

Western Blot Analysis

Sample protein concentrations were measured using the Bio-Rad DC protein assay kit. Proteins were separated and blotted using NuPAGE large protein analysis system (Invitrogen). Total protein (20 μg) was separated on a 3-8% Tris-Acetate Gel at a constant voltage of 150 V for 1 h and the resolved proteins were transferred to a nitrocellulose membrane at 30 V for 1 h. Membrane was blocked in 2% Top-Block (Sigma) in PBS-0.05% Tween-20 (PBST) and then incubated with polyclonal rabbit anti-nNOS antibody (Zymed) and secondary antibody (Donkey anti-rabbit IgG-HRP Santa Cruz) conjugated to horseradish peroxidase in PBST sequentially with 3 wash steps in between. Immunoreactivity was detected using luminal based chemiluminescence detection reagents (Western Lightening, Perkin Elmer) and autoradiography.

[³H] Norepinephrine Release Experiments on the Isolated Right Atrium of the SD Rat

Measurements were performed. Adult SD rats 16-20 weeks old were anesthetized with 4% isofluorane and were killed by cervical dislocation and the right atria were removed and transferred to preheated organ bath containing Tyrode's solution. The atrium was pinned flat between two electrodes. After 45 min equilibration period, the atrium was incubated with 5 μCi [³H] NE for 30 min with field stimulations at 5 Hz for 10 s, then again after a 20 s interval to allow [³H] NE loading. Excess [³H] NE was washed off by superfusing for 60 min at a rate of 4 ml/min with Tyrode's solution. Superfusion was then stopped and the bath solution was replaced every 3 min with a sample being taken on every replacement. The atrium was stimulated for 1 min at 16 min and at 72 min for measuring the [³H] NE release in response to the field stimulation. The radioactivity was measured with a liquid scintillation counter (Tri-carb 2800TR, Packard). [³H] NE outflow was expressed as a percentage of the total radioactivity in the atrium at the time point when the sample was collected.

nNOS Activity Assay

nNOS activity in atria was quantified by measuring the conversion of [³H]-L-arginine to [³H]-L-citrulline using a modification of the procedure described by Bia and Wehling-Henricks [(Bia B L, Cassidy P J, Young M E, Rafael J A, Leighton B, Davies K E, Radda G K, Clarke K. Decreased myocardial nNOS, increased iNOS and abnormal ECGs in mouse models of Duchenne muscular dystrophy. J Mol Cell Cardiol. Oct 1999;31(10):1857-1862), (Wehling-Henricks M, Jordan M C, Roos K P, Deng B, Tidball J G. Cardiomyopathy in dystrophin-deficient hearts is prevented by expression of a neuronal nitric oxide synthase transgene in the myocardium. Hum Mol Genet. Jul. 15 2005;14(14):1921-1933)]. A NOS inhibitor (L-N5-(1-Iminoethyl) ornithine, Dihydrochloride (Calbiochem Ltd.) was added to the assay buffer at a concentration of 10 μg/assay. Scintillation counts were normalized to total protein of the homogenate as determined by measuring the absorbance at 280 nm and expressed as a percent of control values. The results are expressed in fmol citrulline/mg protein per min.

Animal Care

The investigation conformed to the Guide for the Care and Use of Laboratory Animals published by the US National Institutes of Health (NIH Publication No. 85-23, revised 1996), and the Animals (Scientific Procedures) Act 1986 (UK) and were performed under British Home Office license requirements (PPL 30/2130). Age-matched (16-24 weeks old) male spontaneously hypertensive and Wistar-Kyoto (WKY) rats were housed under standard laboratory conditions.

Isolated Rat Sino-Atrial Node/Riglit Vagus Nerve Preparation-Assessment of Vagal Function

Dissection

Animals were anaesthetised with 4% halothane and the carotid vessels cut leading to death by exsanguination. The heart was exposed and the ventricles removed, allowing the atria to be back perfused with 10 ml of heparinised (1,000 U/ml) Tyrode's solution. The thorax and mediastinum were rapidly removed and placed in oxygenated (95% O₂, 5% CO₂) Tyrode's solution at room temperature in a perspex dissecting dish with a Sylgard base. The atria and right vagus were carefully separated and tied off.

Experimental Preparation

Sutures (Ethicon, 5/0 mersilk) were placed at the lateral edges of both atria and the preparation was transferred to a pre-heated (37±0.1° C.) water-jacketed organ bath containing 100 ml of continuously oxygenated Tyrode's solution. The atria were vertically mounted with the suture in the left atrium connected to a stainless steel hook and the suture in the right atrium attached to an isometric force transducer (Harvard Apparatus, Model 60-2997, Mass. USA) connected to an amplifier. Heart rate was triggered from contraction and recorded in real time (Biopac MP100 with Acqknowledge software).

Protocols

Preparations were equilibrated in Tyrode's solution for 60-90 minutes at 37° C. until a stable baseline heart rate was achieved. The right vagus was placed through a pair of custom-built platinum ring electrodes and stimulated at 3, 5, 7 and 10 Hz (15 V, 1 ms pulse duration; order of stimulations randomised) for 25 seconds, with an interval of at least 1 minute between successive stimulations. In some experiments vagal stimulation was repeated after application of the NO donor sodium nitroprusside (SNP; 20 μmol/L, 10 minutes incubation; Sigma). In addition, muscarinic responsiveness of atrial preparations was assessed using cumulative concentration-response curves to carbachol (0.1-0.5 μmol/L; Sigma; 2 minutes incubation at each concentration).

Measurement of ACh Release

Experimental Preparation

Animals were killed and the right atria removed as described above. The preparation was then transferred to a preheated (37 ??0.2 ?C), continuously oxygenated, water-jacketed organ bath containing 4 ml of Tyrode's solution where the atrium was pinned flat between two parallel silver stimulating electrodes 10 mm apart. Our methodology was similar to that described previously₂₈. After a 45 minutes equilibration period (where the Tyrode's solution was replaced every 15 minutes), the atrium was stimulated at 5 Hz (15V, 1 ms pulse duration) for one minute and then again after another minute to stimulate acetylcholine turnover. The preparation was then incubated for 30 minutes with [₃H]choline chloride (10 μCi, Amersham UK) during which the atrium was stimulated at 5 Hz for 10 seconds every 30 seconds to incorporate the radiolabelled choline into the parasympathetic transmitter stores. Tyrode's solution containing 50 μmol/L hemicholinium-3 (Sigma) was used after the incubation period to reduce re-uptake of radioactively labeled transmitter. Excess [₃H]choline was washed from the preparation by superfusing for 60 minutes at a rate of 3 ml/min with Tyrode's solution.

Protocol

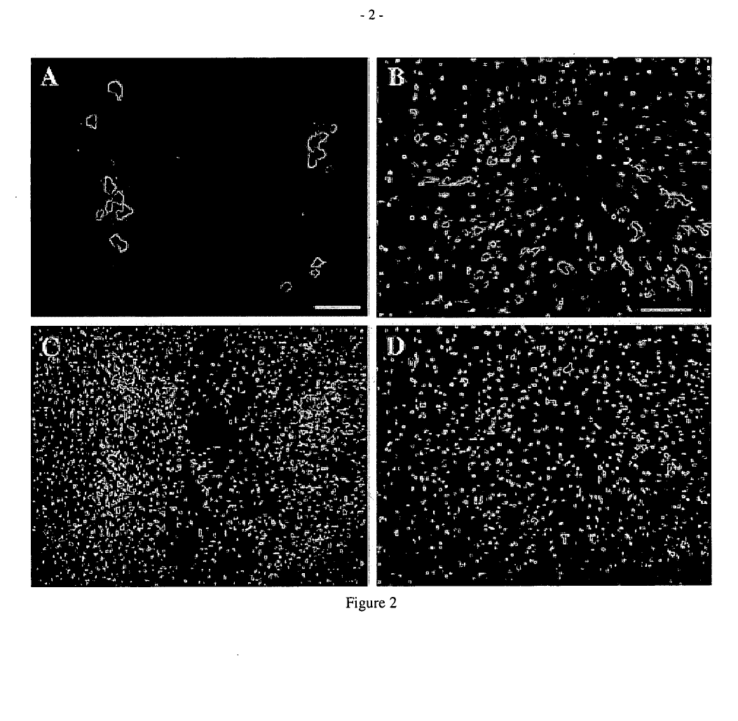

Following the wash period superfusion was stopped and the bath solution replaced every 3 minutes with a 0.5 ml sample being taken on every change of solution. This sample was added to 4.5 ml of scintillation fluid (Ecoscint A, National Diagnostics) and the amount of radioactivity in each sample (disintegrations per minute) measured using a liquid scintillation counter (Tri-carb 2800TR, Packard). After 16 and 94 minutes the atrium was stimulated at 5 Hz for one minute, and after 34 and 112 minutes stimulated again at 10 Hz for one minute (FIG. 2A). In some experiments the soluble guanylate cyclase (sGC) inhibitor 1H-(1,2,4)oxadiazolo(4,3-a)quinoxaline-1-one (ODQ, 10 μmol/L; Sigma) was introduced to the solution following the first (control) 5 Hz stimulation and allowed to incubate for 45 minutes before the second 5 Hz stimulation was performed, while in additional experiments SNP (20 μmol/L; Sigma) was added and allowed to incubate for 15 minutes before the second stimulation. At the end of the experiment, the atrium was immersed overnight in Tyrode's solution containing 4 U/ml papain (Sigma) and the radioactivity contained in the extract determined. [₃H] outflow was expressed as a percentage of the total radioactivity in the atrium at the end of the experiment and that released after superfusion.

Measurement of Right Atrial cGMP Concentration

Experimental Preparation

Isolated, perfused, beating atria were prepared by previously described method_{33,34}. In brief, the right rat atrium was dissected from the heart after the animal was killed. A cannula containing 2 small catheters sealed within it was inserted into the atrium and secured by ligatures. The outer tip of the atrial cannula was open to allow for outflow. The cannulated atrium was transferred to a preheated (36.5 ??0.2?C), continuously oxygenated, waterjacketed organ chamber, immediately perfused with oxygenated Tyrode's solution by means of a peristaltic pump (0.5 ml/min).

Protocols

The atria were perfused for 60 minutes to stabilize. Then [₃H]inulin (5 μCi, Amersham UK) was introduced to the pericardial fluid 20 minutes before the start of the sample collection to measure translocation of extracellular fluid (ECF). The perfusate was collected at 2 minutes intervals at 4° C. for analyses. Collections were performed during perfusion with Tyrod's solution containing carbachol (0.3 μmol/L) for 10 minutes after a 20 minutes control collection period, and again following 10 minutes wash-out with Tyrode's solution.

Measurement of ECF Translocation

The radioactivity of [³H]inulin in atrial perfusate was measured with a liquid scintillation counter, and the amount of ECF translocated through the atrial wall was caculated, the amount of ECF translocated through the atrial wall was calculated as: ECF translocated (μl/min/g atrial wet wt)=total radioactivity in the perfusate (cpm/min)×1000/radioactivity in the pericardial reservoir (cpm/μl)×atrial wet wt (mg).

Radioimmunoassay of cGMP Concentration

For measurement of cGMP concentration in perfusate, 500 μl of the perfusate was treated with trichloroacetic acid to a final concentration of 6% for 15 minutes at room temperature and centrifuged at 4° C. The supernatant (200 μl) was extracted with water-saturated ether three times and then dried using a SpeedVac concentrator (Savant). The dried samples were resuspended and a ₁₂₅I-cGMP radioimmunoassay kit (Amersham UK) was used to measure the amount of cGMP, after the bound form was separated from the free form by magnetic separation. The amount of cGMP efflux was expressed as pmol cGMP/min/g atrial tissue. The molar concentration of cGMP in the interstitial space fluid₃₆ was calculated as cGMP efflux concentration (nmol/L)=cGMP (in pmol/min/g)/ECF translocated (in μl/min/g)×1000.

Right Atrial nNOS Gene Transfer and in vivo Assessment of Cardiac Vagal Responsiveness

Gene Transfer Procedure

SHRs underwent gene transfer via percutaneous injection into the right atrium, using methods similar to those described previously in the guinea pig.₃₂ Animals were anaesthetised with halothane (3-4% for induction and 2-3% for maintenance, in 100% O₂) and injected with 5×10₁₀ particles of replication deficient adenoviral vector encoding nNOS (Ad.nNOS) or enhanced green fluorescent protein (Ad.eGFP; control vector) in sterile phosphate-buffered saline (300 μl injectate volume). The injection was performed using a 26 G needle, placed through the 3_{rd} intercostal space on the right hand side of the animal and directed towards the left axilla. Localisation of the tip of the needle within the right atrial chamber was confirmed prior to injection by flashback of blood into the syringe, and the injection was performed during withdrawal of the needle from the atrial cavity. Phenotyping of transfected animals was performed ˜5 days post-injection.

Anaesthesia and Surgery

Surgical anaesthesia was induced and maintained using halothane as described above, and a tracheostomy was performed to facilitate artificial ventilation (Harvard Rodent Ventilator, Model 683). Following this, the left carotid artery and right jugular vein were cannulated (3 FG and 2 FG respectively, Portex) for recording of blood pressure (SensoNor 840 pressure transducer) and infusion of fluids (4% dextran in 0.9% NaCl; Gentran, Baxter Healthcare Ltd.) and drugs respectively. In addition, subcutaneous stainless steel needle electrodes were placed for recording of the ECG. Heart rate was triggered from the blood pressure and ECG records and displayed in real time using a Biopac Systems MP100 data acquisition system (Biopac Systems Inc) and Acqknowledge software.

Intensive Care

Body temperature was monitored using a rectal thermocouple, and heating lamps placed above and below the animal were used to maintain body temperature within the range 37-38° C. Arterial blood samples were regularly taken in to pre-heparinised capillary tubes and used to measure blood gases and pH (ABL505, Radiometer Copenhagen); alteration of ventilatory parameters and/or infusion of 4.2% sodium bicarbonate solution (in 0.9% NaCl) was used to maintain blood gases and pH within acceptable limits (PaO₂>100 mmHg; PaCO₂ 35-45 mHg; pH 7.4±0.02).

Experimental Protocol

Animals were bilaterally vagotomised and the distal end of the right vagus was placed over a pair of hooked platinum stimulating electrodes. Vagal nerve stimulation was performed for 30 seconds at 3, 5, 7, and 10 Hz (15 V, 3 ms pulse duration; order of stimulations randomised), with an interval of at least one minute between successive stimulations. Rats were euthanised using an intravenous overdose of sodium pentobarbitone (Sagatal; Rhóne Merieux Ltd.) on completion of the experimental protocol.

Measurement of Soluble Guanylate Cyclase and nNOS Protein and nNOS Activity

Western blotting for sGC and nNOS in right atria were performed using standard techniques using commercially available polyclonal antibodies to α₁₋ₛGC (Sigma), nNOS (Zymed Laboratories Inc) and β-actin (Abcam plc) and the Western Lightening detection system (Perkin Elmer Life Sciences). Protein levels were expressed as a ratio of the optical densities of the nNOS/α₁₋ₛGC bands and the β-actin band to control for protein loading. Aorta and forebrain were used to be a positive control for α₁₋ₛGC₂₆ and nNOS₃₇ respectively. 30 μg of protein was loaded into each lane. NOS activity in atria was quantified by measuring the conversion of [₃H]-L-arginine to [₃H]-L-citrulline using a modification of the procedure as previously described._{38,39} Frozen atria were homogenized at 4° C. in 200 μl of 50 mmol/L Tris pH 7.5/containing 1 mmol/L ethylenediamine tetraacetic acid (EDTA), 1 mmol/L ethylene glycol-bis (β-amino ether)-N,N,N′,N′-tetraacetic acid (EGTA), 1 mmol/L DTT and protease inhibitor cocktail (Sigma). After centrifuging the homogenate at 6000 g for 5 minutes at 4° C., 50 μl of the supernatant was incubated in 200 μl of reaction buffer with a final concentration of 50 mmol/L Tris pH 7.5, 5 mmol/L CaCl₂, 1 mmol/L MgCl₂, 14 μmol/L tetrahydrobiopterin, 10 μg/ml calmodulin, 4 μmol/L flavin adenine dinucleotide, 4 μmol/L flavin adenine mononucleotide, 1 mmol/L reduced nicotinamide adenine dinucleotide phosphate (NADPH) and 1 μl of 1 mCi/ml [₃H]-L-arginine. The activities of the nNOS isoforms were measured using specific eNOS inhibitor (L-N₅-(1-Iminoethyl) ornithine, Dihydrochloride, Calbiochem Ltd.) added to the assay buffer at a concentration of 10 μg/assay. After a 30 minutes incubation at 37° C., the reactions were stopped with 20 mmol/L sodium acetate pH 5.5, 0.2 mmol/L EGTA, 1 mmol/L L-citrulline and 2 mmol/L EDTA and poured over Dowex AG-50W-X8 columns (Bio-Rad) previously [₃H]-L-citrulline was eluted with 2 ml of deionized water, and radioactivity was quantified by liquid scintillation counting. The results are expressed in fmol citrulline/mg protein/min.

Solutions and Drugs

Rat Tyrode's solution contained (in mmol/L): NaCl 120, KCl 4.7, MgSO_{4 1.2}, KH₂PO₄ 1.2, NaHCO₃ 25, CaCl₂ 2 and glucose 11. The solution was constantly aerated with carbogen to maintain pH at 7.4. All solutions were prepared fresh on the day of use using deionised water obtained from an Elga water purification system. Experiments using SNP were performed in a darkened room due to the light sensitivity of this drug.

Statistical Analysis

Data are presented as mean±SEM. Differences in the data were assessed using the t-test or Mann-Whitney Rank Sum test as appropriate (SigmaStat, Systat Software Inc). Statistical significance was accepted at p<0.05.

### EXAMPLE 1

### Noradrenergic Neuron-Specific Overexpression of nNOS

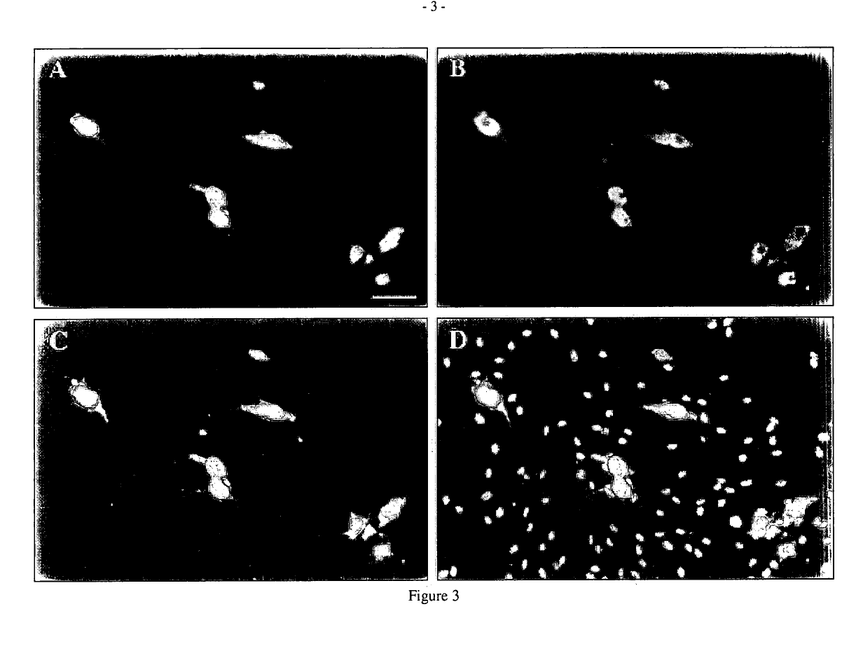

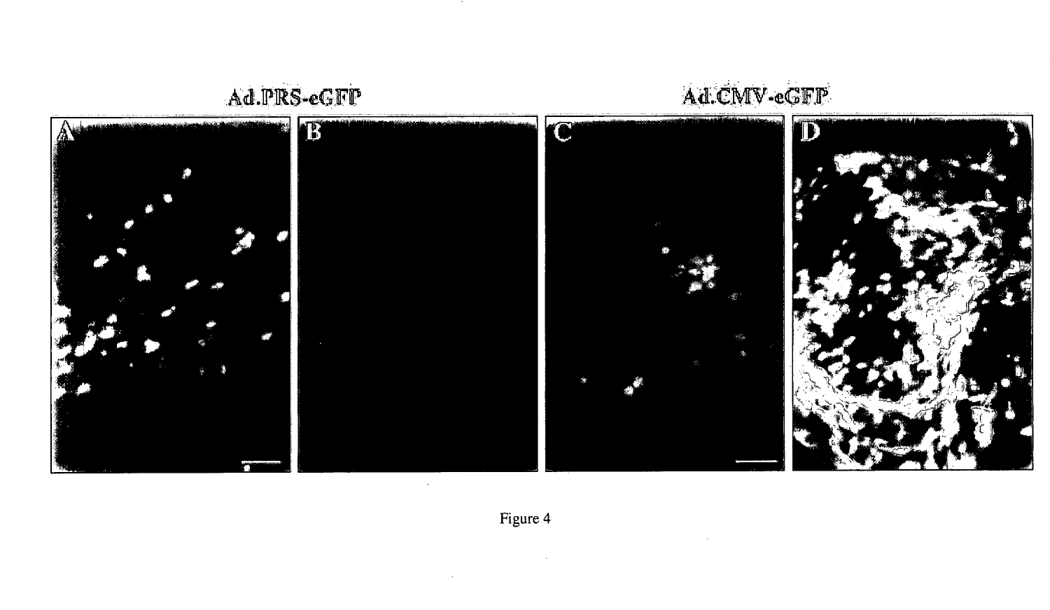

A distinct difference in GFP expression between ADPRS×8-eGFP and AD-CMV-eGFP was observed when the sympathetic neurons from stellate ganglia were transduced with the same amount of two different adenovectors in the same amount of cells (FIG. 2). CMV promoter drove GFP expression in all the cells including non-neurons while the adenovector with PRS×8 promoter selectively transduced sympathetic neurons only. Neuron body, axon and dendrites were clearly identifiable by GFP expression. Immunohistochemistry confirmed that the transduction of sympathetic neurons with Ad. PRS-eGFP resulted in exclusive expression of eGFP in TH positive neurons. The eGFP signal was very strong in both the cell bodies and the axons (FIG. 3) and there was no evidence of leakage into other cell types as indicated by lack of eGFP expression as visualized by DAPI staining (FIG. 3D). The noradrenergic specificity of Ad.PRS-eGFP transduction was further confirmed by the negative control showing no expression of eGFP in intracardiac cholinergic neurons identified by CHAT stained atria, whereas Ad.CMV-eGFP transduced atria showed widespread transduction in CHAT positive cells and other cells types (FIG. 4).

### EXAMPLE 2

### nNOS Gene Transfer Decreases Cardiac Sympathetic Neurotransmission

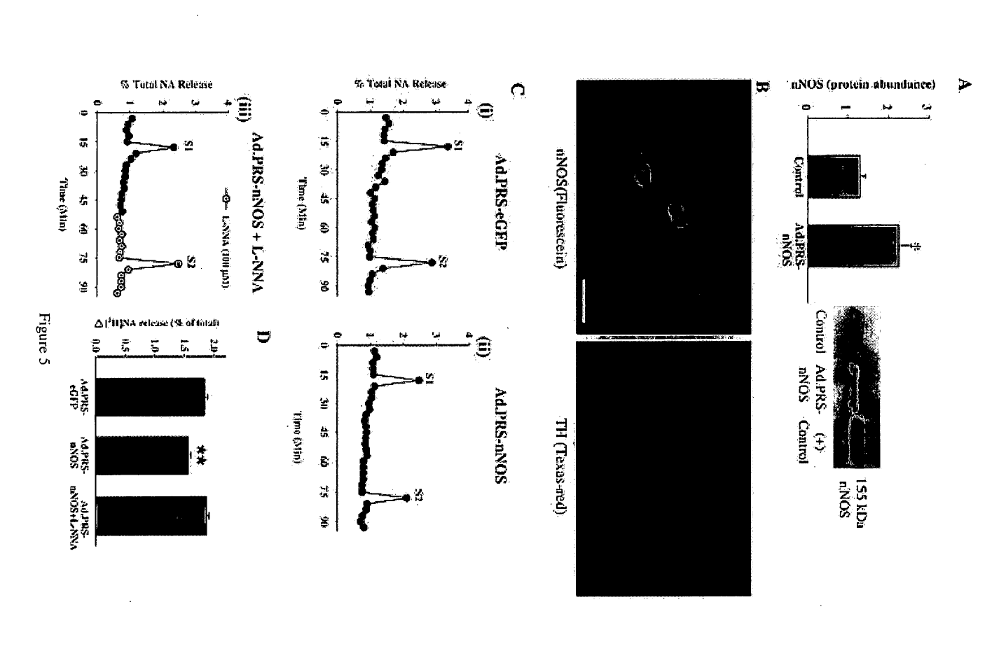

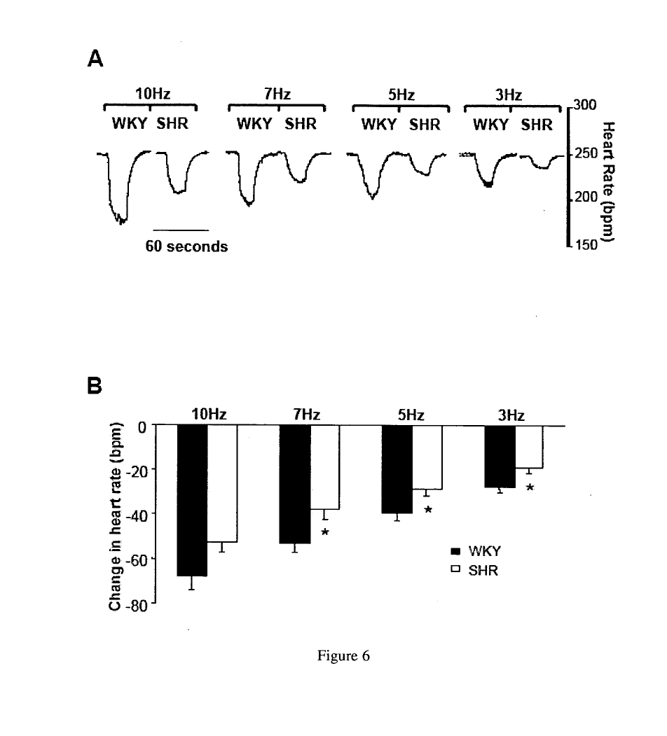

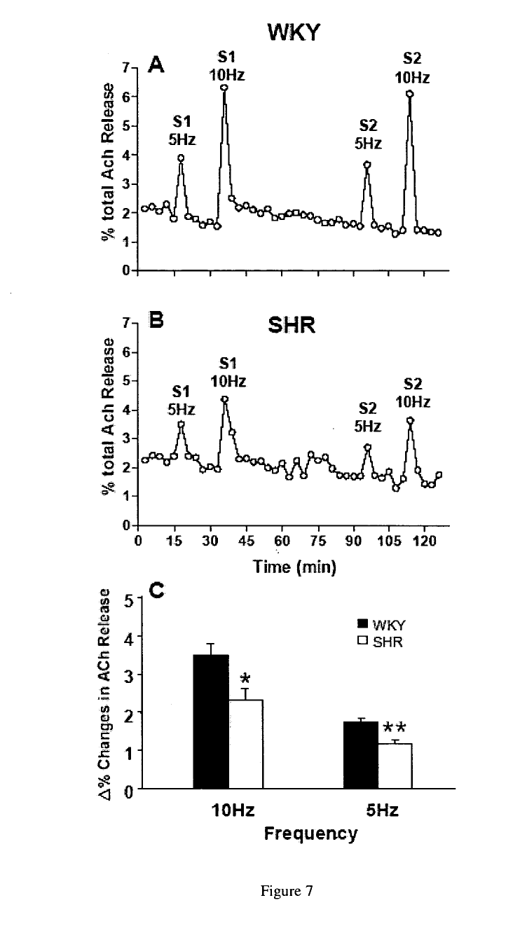

Gene transfer of Ad.PRS-nNOS increased nNOS protein expression compared to nontranduced neurons (FIG. 5A). It also caused significant nNOS expression in tyrosine hydroxylase positive neurons (FIG. 5B) that was associated with an 18.9% increase in nNOS activity from 15.54±1.07 fmol/mg/min in Ad.PRS-eGFP (n=6) to 18.48±1.00 fmol/mg/min in Ad.PRS.nNOS (n=6) treated atria (P=0.03). Ad.PRS-nNOS (n=15) caused 15.2% reduction (from 1.844±0.057 to 1.564±0.048% of total, P<0.01) in evoked NE release compared to Ad. PRS-eGFP (n=11) treated tissue (FIG. 5C). Pretreatment of atria with the NOS inhibitor N_{?}-Nitro-L-arginine (L-NNA) (100 μM, n=6, P<0.01) prevented the Ad.PRS-nNOS-induced attenuation of NE release, and brought it to the level similar to that in Ad.PRS-eGFP-transfected group (FIG. 5D). For comparison, superfusion with NO donor sodium nitroprusside (SNP, 20 μM, n=6) decreased NE release in naive hearts by 21.4% (from 1.631±0.090 to 1.281±0.105% of total, P<0.01).

### EXAMPLE 3

### Cardiac Gene Transfer with nNOS into Sympathetic Nerves Reverses Abnormal Neurotransmission

Sympathetic hyper-responsiveness seen in hypertension may result from oxidative stress impairing the nitric oxide (NO)—cGMP pathway. The hypothesis that gene transfer with neuronal NO synthase (nNOS) restores sympathetic balance in the spontaneously hypertensive rat (SHR) was therefore tested. Percutaneous gene transfer to the right atrial wall was performed in 16-20 weeks old male SHRs and Wistar-Kyoto (WKY) rats, using 5×10¹⁰ particles of adenovirus constructed with a noradrenergic neuron-specific promoter (PRS×8) encoding nNOS (Ad.PRS-nNOS) or enhanced green fluorescence protein (Ad.PRS-eGFP). Five days after transduction, isolated right atria were removed and evoked [³H]norephinephrine (NE) release, NOS activity and cGMP was measured. Tissue levels of cGMP were significantly reduced in Ad.PRS-eGFP treated SHR (0.37±0.01 pmol/mg protein, n=6) compared to Ad.PRS-eGFP treated WKY atria (0.44±0.02 pmol/mg protein, n=6, p<0.05). In the SHR (Ad.PRS-eGFP treated, n=6) NE release was greater compared to WKY atria (Ad.PRS-eGFP treated, n=5, p<0.05); sGC inhibitor 1H-(1,2,4)oxadiazolo(4,3-a)quinoxaline-1-one (ODQ, 10 μmol/L) did not effect Ad.PRS-eGFP treated SHR. Atria treated with Ad.PRS-nNOS had enhanced nNOS activity when compared to Ad.PRS-eGFP treated atria (SHR increased by 40.08±10.03%; WKY increased by 24.90±8.85%; n=6 in each group). Gene transfer with Ad.PRS-nNOS (n=6) in WKY rats caused a 16.12±3.13% reduction in NE release compared to Ad.PRS-eGFP treated atria (n=5, p<0.01). ODQ significantly enhanced the NE release in Ad.PRS-nNOS or Ad.PRS-eGFP treated WKY atria. Gene transfer with Ad.PRS-nNOS in SHR (n=6) attenuated the NE release by 21.24±3.97% compared to Ad.PRS-eGFP (n=5, p<0.05). This attenuation was reversed by ODQ. Gene transfer with Ad.PRS-nNOS also restored cGMP levels in SHR (0.50±0.05 pmol/mg protein, n=6) to those seen in WKY atria

### EXAMPLE 4

### Pharmacological Manipulation of NO-cGMP Pathway

Effect NO Donor

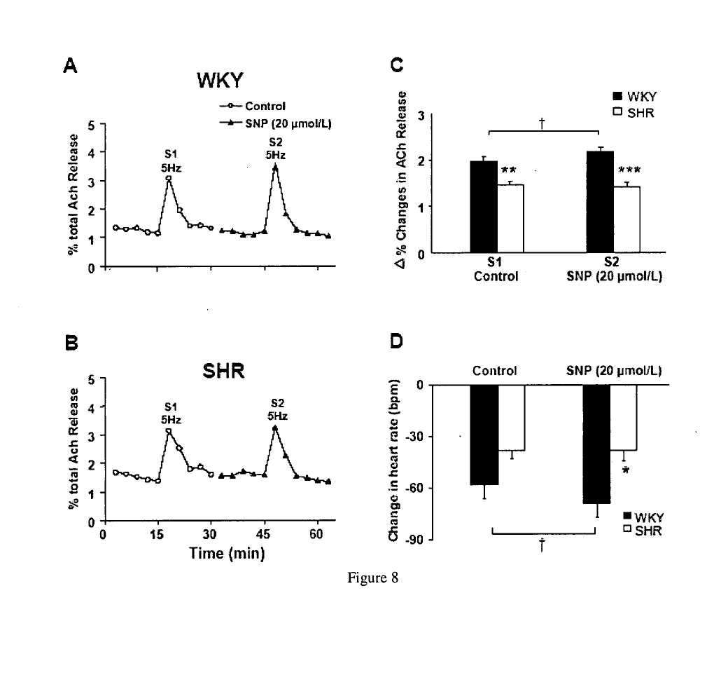

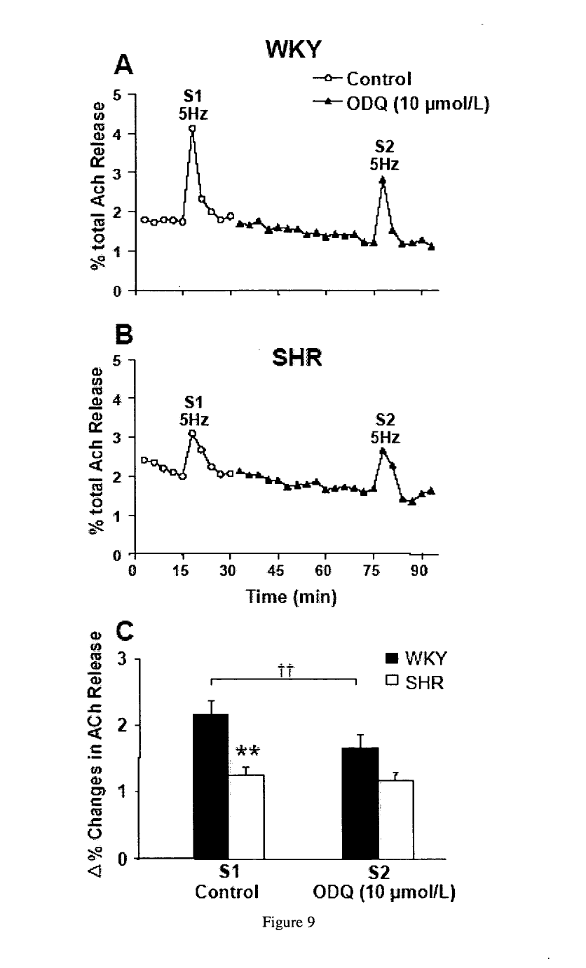

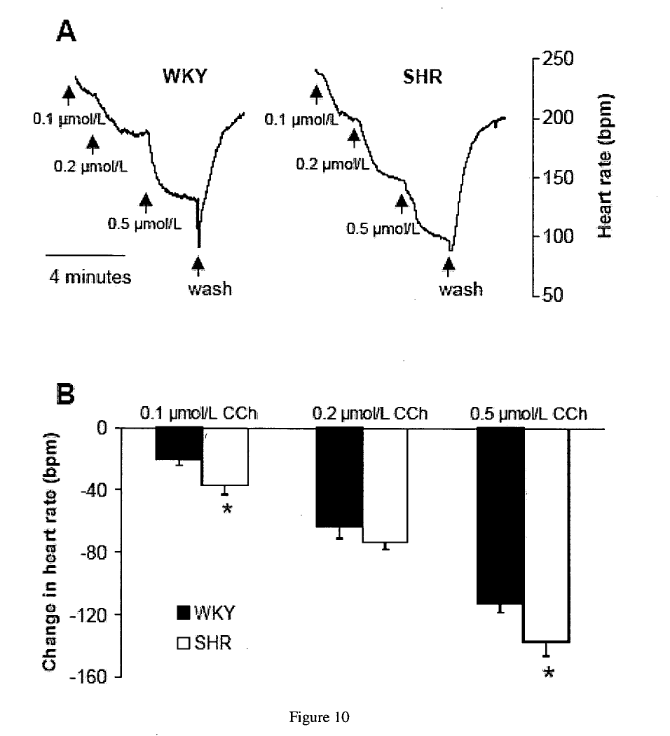

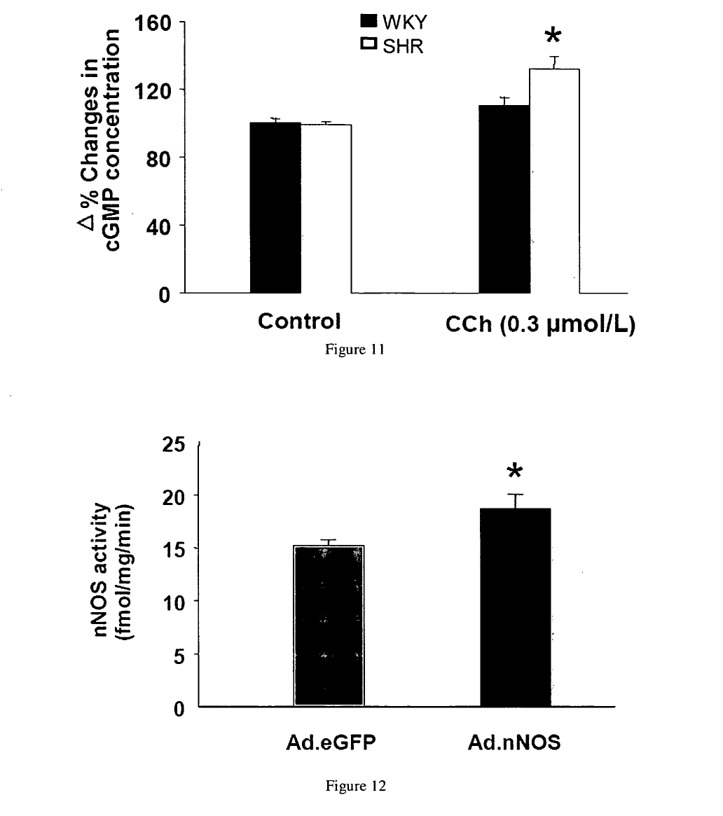

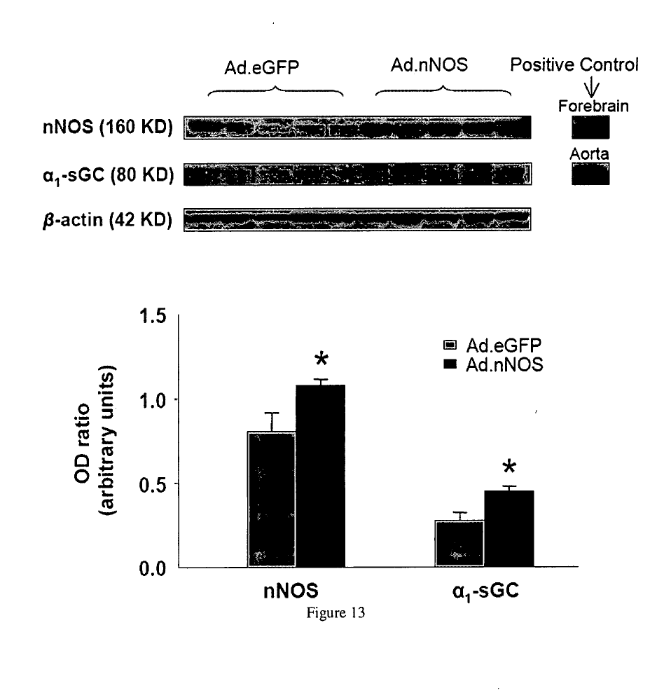

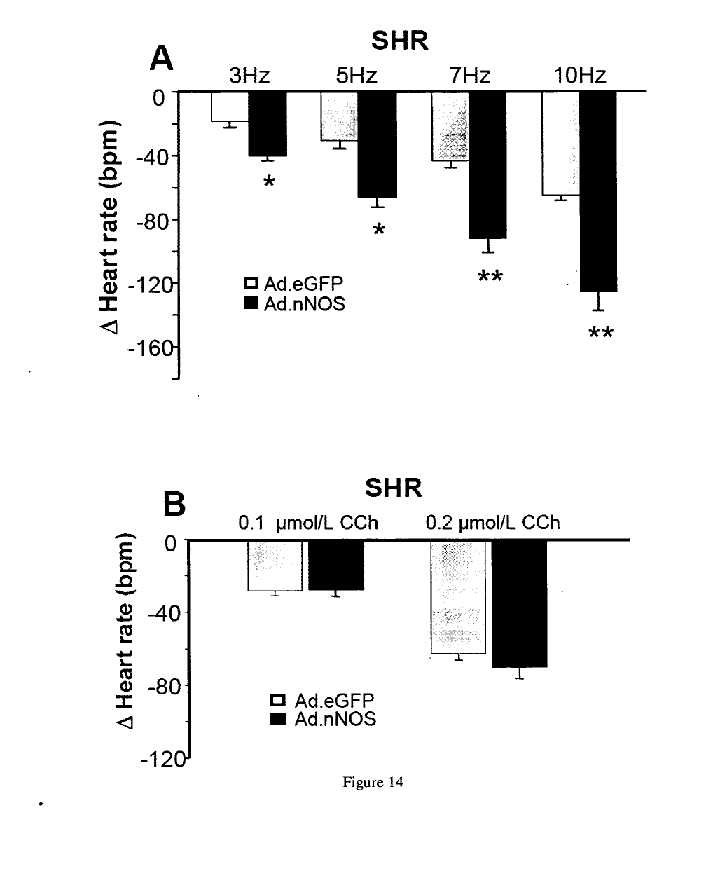

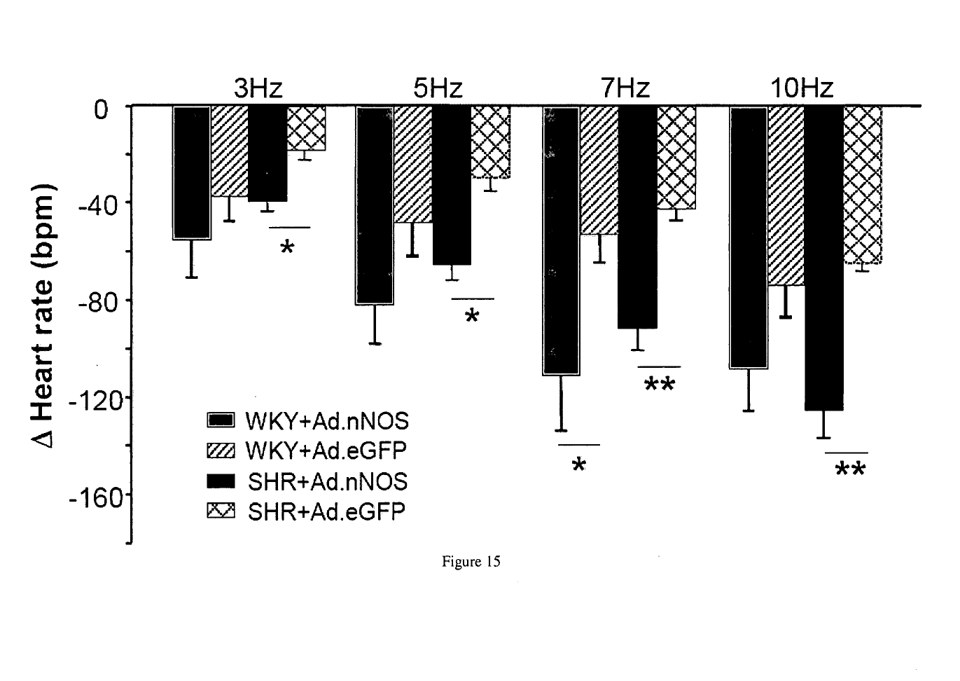

Administration of 20 μmol/L SNP significantly enhanced the release of [³ H]ACh to 5 Hz field stimulated in the WKY (n=8, p<0.05, paired t-test; see FIG. 8A,C), whereas there was no effect in the SHR (n=6, FIG. 8B,C). This translated functionally where SNP significantly enhanced the rate responsiveness to vagal stimulation in the isolated double atrial preparation in the WKY (n=7; p<0.05, paired t-test; FIG. 8D). However, no response was seen in the SHR (n=6) despite a similar increase in basal heart rate in the two strains (WKY: +49±7 (n=8) vs. SHR: +43±6 (n=6) bpm) due the well established action of NO on the pacemaker itself.

What is claimed is:

1. A method of inhibiting or suppressing neurotransmission in a nervous system of a subject, comprising the step of causing a innervation in the subject to overexpress nNOS gene, thereby reducing norepinephrine release, causing inhibition or suppression of neurotransmission.

2. The method of claim 1, whereby the nervous system is a sympathetic nervous system.

3. The method of claim 1, whereby overexpressing the nNOS gene causes increase in nNOS activity.

4. The method of claim 2, whereby the sympathetic innervation is cardiac sympathetic neurons.

5. The method of claim 2, whereby the step of causing a sympathetic innervation of the subject to overexpress nNOS gene is effected by a viral vector.

6. The method of claim 5, whereby the viral vector is an adenoviral vector, lentiviral vector, a retroviral vector, an adeno-associated viral vector, or a combination thereof.

7. A method for producing the viral vector of claim 6 comprising the steps of:
a. introducing into a selected host cell:
(i) a lineraized recombinant shuttle vector comprising: a transcription factors' binding site; followed by a human transcription start site; followed by a nNOS cDNA flanked by a first and second restriction sites; cloned into said first and second restriction sites of a plasmid viral-linker; and
(ii) a viral backbone;
b. transfecting the lineraized shuttle vector and the viral backbone into the host cells, thereby making a recombinant;
c. digesting the recombinant with a restriction enzyme;
d. transfecting the digested recombinant into an embryonic cell; and
e. recovering the virus.

8. The method of claim 7, whereby the linearized recombinant shuttle vector further comprises at least one tandem repeat of the transcription factors' Phox 2a/2b binding site.

9. The method of claim 8, whereby the linearized recombinant shuttle vector comprises between one and eight (8) tandem repeats of the transcription factors' Phox 2a/2b binding site.

10. The method of claim 9, whereby the linearized recombinant shuttle vector comprises at eight (8) tandem repeats of the transcription factors' Phox 2a/2b binding site.

11. The method of claim 7, whereby the selected host cell is a BJ5183 competent cell.

12. The method of claim 7, whereby the first restriction site is SpeI, or XhoI.

13. The method of claim 7, whereby the second restriction site is SpeI, or XhoI.

14. The method of claim 7, whereby the human embryonic cells are AD-293, HEK293 cells or a combination thereof.

15. The method of claim 14, further comprising the step of isolating the recombinant virus prior to the step of recovering, using plaque assay.

16. The method of claim 7, whereby the human transcription start site is human dopamine ?-hydroxilase (hDBH) promoter.

17. The method of claim 7, whereby the restriction enzyme is PacI.

18. The method of claim 7, whereby the viral linker is pAd-PRS-Linker, pTR-Linker or a combination thereof.

19. A method of treating pathological conditions arising due to chronic sympathetic activation in a subject, comprising the step of contacting a sympathetic innervation of the subject with a noradrenergic neuron-specific vector resulting in overexpression of nNOS, thereby decreasing neurotransmission.

20. The method of claim 19, whereby the vector is a viral vector.

21. The method of claim 20, whereby the viral vector is produced by the method of claim 8.

22. The method of claim 19, whereby the sympathetic innervation is cardiac sympathetic neurons.

23. The method of claim 19, whereby the pathological condition due to chronic sympathetic activation is heart failure, hypertension, sudden cardiac death, myocardial infarct or a combination thereof.

24. The method of claim 22, whereby the overexpression of nNOS in the sympathetic innervation of the subject, reduces ?-adrenergic stimulation while maintaining the regulation of sympathetic discharge, thereby meeting cardiac output in response to the subject's activity.

25. A recombinant shuttle vector comprising: a transcription factors' binding site; followed by a human transcription start site; followed by a nNOS cDNA flanked by a first and second restriction sites; cloned into said first and second restriction sites of a plasmid viral-linker.

26. The vector of claim 25, further comprising at least one tandem repeat of the transcription factors' Phox 2a/2b binding site.

27. The vector of claim 25, comprising between one and eight (8) tandem repeats of the transcription factors' Phox 2a/2b binding site.

28. The vector of claim 27, comprising eight (8) tandem repeats of the transcription factors' Phox 2a/2b binding site.

29. The vector of claim 25, wherein the first restriction site is SpeI, or XhoI.

30. The vector of claim 25, wherein the second restriction site is SpeI, or XhoI.

31. The vector of claim 25, wherein the human transcription start site is human dopamine ?-hydroxilase (hDBH) promoter.

32. The vector of claim 25, wherein the viral linker is pAd-PRS-Linker pTR-Linker, or a combination thereof.

33. A composition comprising a noradrenergic neuron-specific vector.

34. The composition of claim 33, wherein said vector is a viral vector.

35. The composition of claim 34, wherein said viral vector comprises a nucleic acid construct comprising a gene encoding nNOS flanked by a first and a second restriction sites whose expression is controlled by a first and a second promoter, such that said gene is expressed in sympathetic nerves and overexpresses nNOS.

36. The composition of claim 35, wherein the first restriction site is SpeI, or XhoI.

37. The composition of claim 35, wherein the second restriction site is SpeI, or XhoI.

38. The composition of claim 35, wherein the first promoter is a transcription factors' Phox 2a/2b binding site.

39. The composition of claim 35, wherein the second promoter is a human dopamine ?-hydroxilase (hDBH) promoter.

40. The composition of claim 35, further comprising between one and eight (8) tandem repeats of the transcription factors' Phox 2a/2b binding site.

41. The composition of claim 40, comprising eight (8) tandem repeats of the transcription factors' Phox 2a/2b binding site.

42. A method of treating hypertension in a subject, comprising administering to the subject the composition of claim 35, thereby overexressing nNOS in the subjects cardiac sympathetic nerves.

43. The method of claim 42, whereby treating is inhibiting, ameliorating, reducing blood pressure or a combination thereof.

44. The method of claim 42, whereby treating is curing.

45. A method of restoring reduced cardiac vagal activity in a subject, comprising administering to the subject the composition of claim 35, thereby overexpressing nNOS in the cardiac vagus, increasing nitrous oxide concentration and restoring impaired No-cGMP signaling.

46. The method of claim 45, whereby the reduced vagal activity results in hypertension in the subject.

47. The method of claim 45, further comprising the step of administering to the subject an effective amount of sodium nitroprusside (SNP).

48. The method of claim 45, further comprising administering to the subject an effective amount of soluble guanylate cyclase (sGC).

49. The composition of claim 36, further comprising sodium nitroprusside, soluble guanylate cyclase, or their combination.
